(19) United States
(12) Patent Application Publication
(10) Pub. No.: US 2014/0057998 A1
(43) Pub. Date: Feb. 27, 2014

(54) USE OF HIGHLY-BRANCHED POLYCARBONATES IN COSMETIC AND DERMATOLOGICAL FORMULATIONS

(71) Applicants: Volker Wendel, Seeheim-Jugenheim (DE); Matthias Laubender, Schifferstadt (DE); Jean-Francois Stumbe, Strasbourg (FR); Bernd Bruchmann, Freinsheim (DE)

(72) Inventors: Volker Wendel, Seeheim-Jugenheim (DE); Matthias Laubender, Schifferstadt (DE); Jean-Francois Stumbe, Strasbourg (FR); Bernd Bruchmann, Freinsheim (DE)

(73) Assignee: BASF SE, Ludwigshafen (DE)

(21) Appl. No.: 14/065,789

(22) Filed: Oct. 29, 2013

Related U.S. Application Data
(62) Division of application No. 12/952,646, filed on Nov. 23, 2010, now abandoned.
(60) Provisional application No. 61/264,674, filed on Nov. 26, 2009.

(51) Int. Cl. A61K 8/85; A61Q 15/00

(52) U.S. Cl. CPC A61K 8/85; A61Q 15/00
USPC 514/772.1; 514/772.3; 525/461

(57) ABSTRACT

The present invention relates to compositions which comprise highly-branched polycarbonates, to the use of these highly-branched polycarbonates in cosmetics and dermatology and to substituted highly-branched polycarbonates.

The present invention relates to compositions which comprise highly-branched polycarbonates, to the use of these highly-branched polycarbonates in cosmetics and dermatology and to substituted highly-branched polycarbonates.

Thickeners are used to a great degree in the field of pharmacy and cosmetics for increasing the viscosity of preparations.

The thickeners are chosen according to whether the preparation is aqueous, oily or surface-active. An overview on this topic is given in Hugo Janistyn, Handbuch der Kosmetika and Riechstoffe [Handbook of cosmetics and fragrances], Hüthig Verlag Heidelberg, volume 1, 3rd edition, 1978, p. 979.

Examples of thickeners that are often used for aqueous solutions are fatty acid polyethylene glycol monoesters, fatty acid polyethylene glycol diesters, fatty acid alkanolamides, oxyethylated fatty alcohols, ethoxylated glycerol fatty acid esters, cellulose ethers, sodium alginate, polyacrylic acids, and neutral salts.

Polymers comprising carboxyl groups are also known as thickeners. These include homopolymers and copolymers of monoethylenically unsaturated carboxylic acids, such as acrylic acid, methacrylic acid, maleic acid, maleic anhydride and itaconic acid. These polymers are often crosslinked at least to a small extent. Such polymers are described, for example, in U.S. Pat. No. 2,798,053, U.S. Pat. No. 3,915,921, U.S. Pat. No. 3,940,351, U.S. Pat. No. 4,062,817, U.S. Pat. No. 4,066,583, U.S. Pat. No. 4,267,103, U.S. Pat. No. 5,349,030 and U.S. Pat. No. 5,373,044.

Frequent disadvantages of these polymers when used as thickeners are their pH dependency and hydrolytic instability. Furthermore, large amounts of the polymers are often required for achieving the desired thickening effect, and the stability of the preparations in the presence of electrolytes is low.

Naturally occurring materials such as casein, alginates, methylcellulose, hydroxyethylcellulose, hydroxypropylcellulose and carbomethoxycellulose are also used as thickeners. These have, inter alia, the disadvantage of sensitivity to microbiological factors and the addition of biocides is consequently required.

Typical thickeners of oily preparations, also called oil thickeners below, are metal soaps, amorphous silicon dioxide, hydroxystearin, compounds of quaternary ammonium bases with bentonites, waxes and paraffins.

Surfactant solutions are thickened, for example, by fatty acid alkylolamides, amine oxides, cellulose derivates, polysaccharides and the aforementioned polymers comprising carboxyl groups.

It was an object of the present invention to find rheology-modifying, in particular thickening, in particular oil-thickening, polymers which are highly suitable for cosmetic applications and have good application properties especially in the field of skin cosmetics. Besides the good thickening effect for a small use of material, these also include clarity in the case of gel applications, (co-)emulsifying and stabilizing effect for oil-insoluble and/or difficult-to-stabilize components, good incorporability into cosmetic preparations. For gels in particular, the highest possible transparency (clarity) of the preparations is desired. In order to ensure the broadest possible formulatability, it is desired that the thickeners are low-color and low-odor, ideally colorless and odorless. Moreover, for use in (skin) cosmetic and/or dermatological applications, it is necessary that no allergenic reactions are triggered.

The object is achieved by the highly-branched polycarbonates described below.

The preparation of high-functionality highly-branched polycarbonates and their use as adhesion promoters, thixotropic agents or as building blocks for the preparation of polyaddition or polycondensation polymers, for example of paints and varnishes, coatings, adhesives, sealants, castable elastomers or foams, is known from WO 2005/026234.

WO 2006/018063 describes compositions for hair cosmetics which comprise hydrophobically functionalized dendritic macromolecules. The dendritic macromolecules are composed either of polyester units (obtainable under the trade name Boltorn) or of polyamide units (obtainable under the trade name Hybrane).

DE 10 2005 063 096 describes cosmetic compositions which comprise 0.05 to 20% by weight of at least one hyperbranched polyester and/or polyester amide. The compositions reportedly have hair cleansing and/or hair care properties. The polyesters and/or polyester amides are not substituted.

WO 2004/078809 discloses highly-branched polymers and cosmetic compositions comprising these.

Within the context of this invention, highly-branched polycarbonates are understood as meaning uncrosslinked macromolecules with hydroxyl groups and carbonate or carbamoyl chloride groups which are both structurally and also molecularly nonuniform. They can firstly be composed starting from a central molecule analogously to dendrimers, but with nonuniform chain length of the branches. They may secondly also be linear in composition, with functional side groups, or else, as a combination of the two extremes, have linear and branched molecular moieties. For the definition of dendrimeric and highly-branched polymers, see also P. J. Flory, J. Am. Chem. Soc. 1952, 74, 2718 and H. Frey et al., Chem. Eur. J. 2000, 6, No. 14, 2499.

In connection with the present invention, “highly branched” is understood as meaning that the degree of branching (DB), i.e. the average number of dendritic linkages plus the average number of end groups per molecule, divided by the sum of the average number of dendritic linkages, the average number of linear linkages and the average number of end groups, multiplied by 100, is 10 to 99.9%, preferably 20 to 99%, particularly preferably 20-95%.

Besides the expression highly branched, the expression hyperbranched is also known from the literature. Within the context of the present invention, the two expressions should be understood synonymously.

In connection with the present invention, “dendrimeric” is understood as meaning that the degree of branching is 99.9-100%. For the definition of the degree of branching, see H. Frey et al., Acta Polym. 1997, 48, 30.

The highly-branched polycarbonates are prepared as described below.

As starting material it is possible to use phosgene, diphosgene or triphosgene, although organic carbonates (A) are preferably used.

The radicals R of the organic carbonates (A) of the general formula RO[(CO)O]ₙR used as starting material are in each case independently of one another a straight-chain or branched aliphatic, aromatic/aliphatic or aromatic hydrocarbon radical having 1 to 20 carbon atoms. The two radicals R may also be joined together to form a ring. It is preferably an aliphatic hydrocarbon radical and particularly preferably a straight-chain or branched alkyl radical having 1 to 5 carbon atoms, or a substituted or unsubstituted phenyl radical.

The carbonates may preferably be simple carbonates of the general formula RO(CO)OR, i.e. in this case, n is 1.

In general, n is an integer between 1 and 5, preferably between 1 and 3.

Dialyl or diaryl carbonates can be prepared, for example, from the reaction of aliphatic, araliphatic or aromatic alcohols, preferably monoalcohols, with phosgene. Furthermore, they can also be prepared via oxidative carbonylation of the alcohols or phenols by means of CO in the presence of noble metals, oxygen or NOₓ. For preparation methods of diaryl or dialkyl carbonates, also see Ullmann's Encyclopedia of Industrial Chemistry, 6th edition, 2000 Electronic Release, Verlag Wiley-VCH.

Examples of suitable carbonates comprise aliphatic, aromatic/aliphatic or aromatic carbonates, such as ethylene carbonate, 1,2- or 1,3-propylene carbonate, diphenyl carbonate, ditolyl carbonate, dixylyl carbonate, dinaphthyl carbonate, ethylphenyl carbonate, dibenzyl carbonate, dimethyl carbonate, diethyl carbonate, dipropyl carbonate, dibutyl carbonate, diisobutyl carbonate, dipentyl carbonate, dihexyl carbonate, dicyclohexyl carbonate, diheptyl carbonate, dioctyl carbonate, didecyl carbonate or didodecyl carbonate.

Examples of carbonates in which n is greater than 1 comprise dialkyl dicarbonates, such as di(tert-butyl)dicarbonate or dialkyl tricarbonates, such as di(tert-butyl)tricarbonate.

Preference is given to using aliphatic carbonates, in particular those in which the radicals comprise 1 to 5 carbon atoms, such as, for example, dimethyl carbonate, diethyl carbonate, dipropyl carbonate, dibutyl carbonate or diisobutyl carbonate or diphenyl carbonate as aromatic carbonate.

The organic carbonates are reacted with at least one aliphatic or aromatic alcohol (B) which has at least 3 OH groups, or mixtures of two or more different alcohols.

Examples of compounds with at least three OH groups comprise glycerol, trimethylolmethane, trimethylolethane, trimethylolpropane, 1,24-butanetriol, tris(hydroxymethyl)amine, tris(hydroxyethyl)amine, tris(hydroxypropyl)amine, pentaerythritol, diglycerol, triglycerol, polyglycerols, bis(trimethylolpropane), tris(hydroxymethyl)isocyanurate, tris(hydroxyethyl)isocyanurate, phloroglucinol, trihydroxytoluene, trihydroxydimethylbenzene, phloroglycides, hexahydroxybenzene, 1,3,5-benzenetrimethanol, 1,1,1-tris(4′-hydroxyphenyl)methane, 1,1,1-tris(4′-hydroxyphenyl)ethane, sugars, such as, for example, glucose, sugar derivatives, tri- or higher-functional polyetherols based on tri- or higher-functional alcohols and ethylene oxide, propylene oxide or butylene oxide or mixtures thereof, or polyesterols. Here, particular preference is given to glycerol, trimethylolethane, trimethylolpropane, 1,2,4-butanetriol, pentaerythritol, and polyetherols thereof based on ethylene oxide or propylene oxide.

These polyfunctional alcohols can also be used in a mixture with difunctional alcohols (B′), with the proviso that the average OH functionality of all of the alcohols used is together greater than 2. Examples of suitable compounds with two OH groups comprise ethylene glycol, diethylene glycol, triethylene glycol, 1,2- and 1,3-propanediol, dipropylene glycol, tripropylene glycol, neopentyl glycol, 1,2-, 1,3- and 1,4-butanediol, 1,2-, 1,3- and 1,5-pentanediol, hexanediol, cyclopentanediol, cyclohexanediol, cyclohexanedimethanol, bis(4-hydroxycyclohexyl)methane, bis(4-hydroxycyclohexyl)ethane, 2,2-bis(4-hydroxycyclohexyl)propane, 1,1′-bis(4-hydroxyphenyl)-3,3,5-trimethylcyclohexane, resorcinol, hydroquinone, 4,4′-dihydroxydiphenyl, bis(4-hydroxyphenyl)sulfide, bis(4-hydroxyphenyl)sulfone, bis(hydroxymethyl)benzene, bis(hydroxymethyl)toluene, bis(p-hydroxyphenyl)methane, bis(p-hydroxyphenyl)ethane, 2,2-bis(p-hydroxyphenyl)propane, 1,1-bis(p-hydroxyphenyl)cyclohexane, dihydroxybenzophenone, difunctional polyetherpolyols based on ethylene oxide, propylene oxide, butylene oxide or mixtures thereof, polytetrahydrofuran, polycaprolactone or polyesterols based on diols and dicarboxylic acids.

The diols serve for the fine adjustment of the properties of the polycarbonate. If difunctional alcohols are used, the ratio of difunctional alcohols (B′) to the at least trifunctional alcohols (B) is determined by the person skilled in the art according to the desired properties of the polycarbonate. Generally, the amount of the alcohol or alcohols (B′) is 0 to 39.9 mol %, with regard to the total amount of all of the alcohols (B) and (B′) together. Preferably, the amount is 0 to 35 mol %, particularly preferably 0 to 25 mol % and very particularly preferably 0 to 10 mol %.

The high-functionality highly-branched polycarbonates are terminated after the reaction, i.e. without further modification, with hydroxyl groups and/or with carbonate groups or carbamoyl chloride groups. They are readily soluble in various solvents, for example in water, alcohols, such as methanol, ethanol, butanol, alcohol/water mixtures, acetone, 2-butanone, ethyl acetate, butyl acetate, methoxypropyl acetate, methoxyethyl acetate, tetrahydrofuran, dimethylformamide, dimethylacetamide, N-methylpyrrolidone, ethylene carbonate or propylene carbonate.

Within the context of this invention, a high-functionality polycarbonate is to be understood as meaning a product which, besides the carbonate groups which form the polymer backbone, has, terminally or laterally, also at least three, preferably at least six, more preferably at least ten, functional groups. The functional groups are carbonate groups or carbamoyl chloride groups and/or OH groups. The number of terminal or lateral functional groups is in principle not limited upwards, but products with a very high number of functional groups may have undesired properties, such as, for example, high viscosity or poor solubility. The high-functionality polycarbonates of the present invention in most cases have not more than 500 terminal or lateral functional groups, preferably not more than 100 terminal or lateral functional groups.

The simplest structure of the condensation product (K), illustrated using the example of the reaction of a carbonate (A) with a di- or polyalcohol (B), produces here the arrangement XYₘ or YₘX, where X is a carbonate group, Y is a hydroxyl group and m is generally an integer between 1 and 6, preferably between 1 and 4, particularly preferably between 1 and 3. The reactive group which results here as an individual group is generally referred to below as “focal group”.

If, for example, in the preparation of the simplest condensation product (K) from a carbonate and a dihydric alcohol the reaction ratio is 1:1, the result is on average a molecule of the type XY, illustrated by the general formula 1.

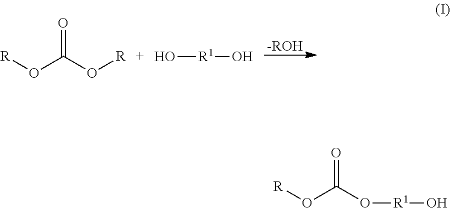

In the case of the preparation of the condensation product (K) from a carbonate and a trihydric alcohol with a reaction ratio of 1:1 the result is on average a molecule of the type XY₂, illustrated by the general formula 2. The focal group here is a carbonate group.

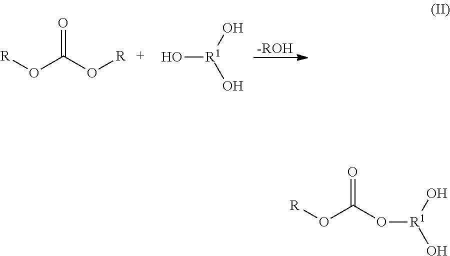

In the preparation of the condensation product (K) from a carbonate and a tetrahydric alcohol, likewise with the reaction ratio of 1:1, the result is on average a molecule of the type XY₃, illustrated by the general formula 3. The focal group here is a carbonate group.

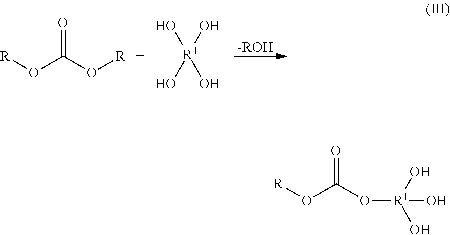

In the formulae 1 to 3, R has the meaning defined at the outset and R¹ is an aliphatic or aromatic radical.

Furthermore, the preparation of the condensation product (K) can take place, for example, also from a carbonate and a trihydric alcohol, illustrated by the general formula 4, in which case the molar reaction ratio is 2:1. Here, the result is on average a molecule of the type X₂Y, the focal group here being an OH group. In the formula 4, R and R¹ have the same meaning as in the formulae 1 to 3.

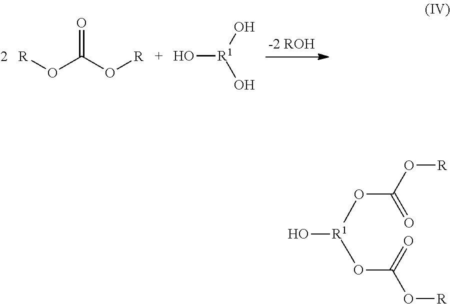

If additionally difunctional compounds, e.g. a dicarbonate or a diol, are added to the components, then this brings about an extension of the chains, as illustrated, for example, in the general formula 5. The result is again on average a molecule of the type XY₂, and the focal group is a carbonate group.

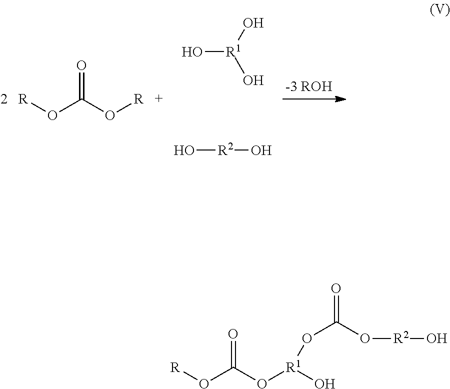

In formula 5, R² is an aliphatic or aromatic radical, R and R¹ are defined as described above.

It is also possible to use two or more condensation products (K) for the synthesis. In this case, it is possible on the one hand to use two or more alcohols and/or two or more carbonates. Furthermore, through the selection of the ratio of the alcohols and the carbonates used, and/or of the phosgenes, it is possible to obtain mixtures of different condensation products with a different structure. This may be illustrated by way of example using the example of the reaction of a carbonate with a trihydric alcohol. Using the starting materials in the ratio 1:1, as shown in (II) thus gives a molecule XY₂. Using the starting materials in the ratio 2:1, as depicted in (IV), thus gives a molecule X₂Y. In the case of a ratio between 1: 1 and 2:1, a mixture of molecules XY₂ and X₂Y is obtained.

The simple condensation products (K) described by way of example in the formulae 1-5 preferably undergo intermolecular reaction to form high-functionality polycondensation products, referred to below as polycondensation products (P). The reaction to give the condensation product (K) and to the polycondensation product (P) usually takes place at a temperature from 0 to 300° C., preferably 0 to 250° C., particularly preferably at 60 to 200° C. and very particularly preferably at 60 to 160° C. without a diluent or in solution. Here, it is generally possible to use any solvents which are inert towards the respective starting material. Preference is given to using organic solvents, such as, for example, decane, dodecane, benzene, toluene, chlorobenzene, xylene, dimethylformamide, dimethylacetamide or solvent naphtha.

In one preferred embodiment, the condensation reaction is carried out without a diluent. The monfunctional alcohol released during the reaction or the phenol ROH can be removed from the reaction equilibrium, for example by distillation, where appropriate under reduced pressure, in order to increase the rate of the reaction.

If distillative removal is envisaged, it is generally advisable to use carbonates which, during the reaction, liberate alcohols or phenols ROH with a boiling point of less than 140° C. under the prevailing pressure.

In order to increase the rate of the reaction it is also possible to add catalysts or mixtures of catalysts. Suitable catalysts are compounds which catalyze the esterification or transesterification reactions, for example alkali metal hydroxides, alkali metal carbonates, alkali metal hydrogencarbonates, preferably those of sodium, potassium or cesium, tertiary amines, guanidines, ammonium compounds, phosphonium compounds, organoaluminum, organotin, organozinc, organotitanium, organozirconium or organobismuth compounds, and also so-called double-metal cyanide (DMC) catalysts, as described, for example, in DE 10138216 or in DE 10147712.

Preference is given to using potassium hydroxide, potassium carbonate, potassium hydrogencarbonate, diazabicyclooctane (DABCO), diazabicyclononene (DBN), diazabicycloundecene (DBU), imidazoles, such as imidazole, 1-methylimidazole or 1,2-dimethylimidazole, titanium tetrabutoxide, titanium tetraisopropoxide, dibutyltin oxide, dibutyltin dilaurate, tin dioctoate, zirconium acetylacetonate or mixtures thereof.

The catalyst is generally added in an amount of from 50 to 10 000, preferably from 100 to 5000 ppm by weight, based on the amount of alcohol or alcohol mixture used.

It is also possible to control the intermolecular polycondensation reaction either by adding the appropriate catalyst, or by selecting an appropriate temperature. Furthermore, the average molecular weight of the polymer (P) can be adjusted via the composition of the starting components and via the residence time.

The condensation products (K) and the polycondensation products (P), which have been prepared at elevated temperature, are usually stable for a relatively long period of time at room temperature.

In view of the nature of the condensation products (K) it is possible for the condensation reaction to result in polycondensation products (P) having different structures, which have branches, but no crosslinks. Furthermore, in an ideal case, the polycondensation products (P) have either one carbonate or carbamoyl chloride group as focal group and more than two OH groups, or else one OH group as focal group and more than two carbonate or carbamoyl chloride groups. The number of reactive groups arises here from the nature of the condensation products (K) used and the degree of polycondensation.

By way of example, a condensation product (K) according to the general formula 2 can react by triple intermolecular condensation to give two different polycondensation products (P), which are shown in the general formulae 6 and 7.

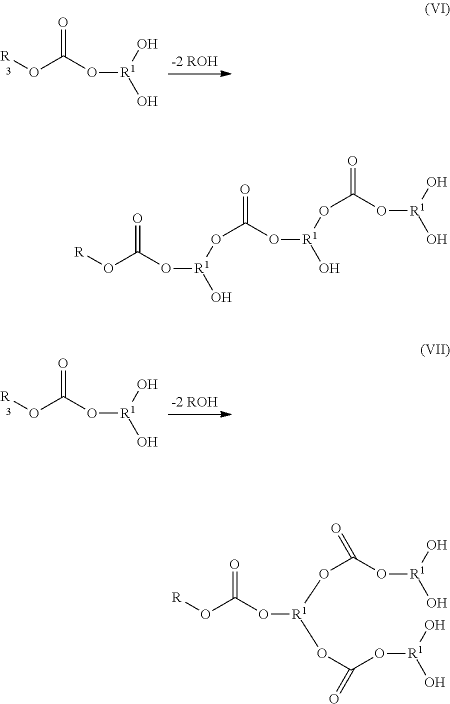

In formula 6 and 7, R and R¹ are as defined above.

There are a variety of options for terminating the intermolecular polycondensation reaction. For example, the temperature can be lowered to a range in which the reaction comes to a standstill and the product (K) or the polycondensation product (P) is storage-stable.

Furthermore, the catalyst can be deactivated; in the case of basic catalysts, for example, by adding an acidic component, for example a Lewis acid or an organic or inorganic protonic acid.

In a further embodiment, as soon as the intermolecular reaction of the condensation product (K) has produced a polycondensation product (P) with the desired degree of polycondensation, the reaction can be terminated by adding to the product (P) a product containing groups that are reactive towards the focal group of (P). For example, in the case of a carbonate group as focal group, for example a monoamine, diamine or polyamine can be added. In the case of a hydroxyl group as focal group, a mono-, di- or polyisocyanate, a compound comprising epoxy groups, or an acid derivative reactive with OH groups can be added to the product (P).

The preparation of the high-functionality polycarbonates according to the invention takes place in most cases within a pressure range from 0.1 mbar to 20 bar, preferably at 1 mbar to 5 bar, in reactors or reactor cascades which are operated batchwise, semicontinuously or continuously.

As a result of the aforementioned setting of the reaction conditions and, where appropriate, through the selection of the appropriate solvent it is possible to further process the products according to the invention, following their preparation, without further purification.

In a further preferred embodiment, the product is stripped, i.e. freed from low molecular weight volatile compounds. For this, when the desired degree of conversion has been reached, the catalyst can optionally be deactivated and the low molecular weight volatile constituents, for example monoalcohols, phenols, carbonates, hydrogen chloride or readily volatile oligomeric or cyclic compounds, can be removed by distillation, if appropriate with introduction of a gas, preferably nitrogen, carbon dioxide or air, and if appropriate under reduced pressure.

In a further preferred embodiment, the polycarbonates according to the invention may acquire further functional groups in addition to the functional groups already acquired as a result of the reaction. The functionalization can take place during molecular weight buildup or else subsequently, i.e. after the end of the actual polycondensation.

If, before or during molecular weight buildup, components are added which have further functional groups or functional elements besides hydroxyl or carbonate groups, then the result is a polycarbonate polymer containing randomly distributed functionalities different from the carbonate, carbamoyl chloride or hydroxyl groups.

Effects of this kind can be achieved, for example, by adding compounds, during the polycondensation, which besides hydroxyl groups, carbonate groups or carbamoyl chloride groups, carry further functional groups or functional elements, such as mercapto groups, primary, secondary or tertiary amino groups, ether groups, carboxylic acid groups or derivatives thereof, sulfonic acid groups or derivatives thereof, phosphonic acid groups or derivatives thereof, silane groups, siloxane groups, aryl radicals or long-chain alkyl radicals. For the modification by means of carbamate groups it is possible, for example, to use ethanolamine, propanolamine, isopropanolamine, 2-(butylamino)ethanol, 2-(cyclohexylamino)ethanol, 2-amino-1-butanol, 2-(2′-aminoethoxy)ethanol or higher alkoxylation products of ammonia, 4-hydroxypiperidine, 1-hydroxyethylpiperazine, diethanolamine, dipropanolamine, diisopropanolamine, tris(hydroxymethyl)aminomethane, tris(hydroxyethyl)aminomethane, ethylenediamine, propylenediamine, hexamethylenediamine or isophoronediamine.

For the modification with mercapto groups it is possible to use, for example, mercaptoethanol. Tertiary amino groups can be produced, for example, through incorporation of triethanolamine, tripropanolamine, N-methyldiethanolamine, N-methyldipropanolamine or N.N-dimethylethanolamine. Ether groups can be generated, for example, by incorporating di- or higher-functional polyetherols by condensation. By adding dicarboxylic acids, tricarboxylic acids, dicarboxylic acid esters, such as, for example, dimethyl terephthalate or tricarboxylic acid esters, it is possible to produce ester groups. By reaction with long-chain alkanols or alkanediols it is possible to introduce long-chain alkyl radicals. The reaction with alkyl or aryl diisocyanates generates polycarbonates containing alkyl, aryl and urethane groups; the addition of primary or secondary amines leads to the introduction of urethane groups or urea groups.

In one preferred embodiment, the highly-branched polycarbonates are completely or partially substituted by linear or branched C₄- to C₄₀-alkyl and/or -alkenyl radicals. Within the context of the present invention, alkenyl radicals may be monounsaturated or polyunsaturated.

Within the scope of the present invention, substitution means that the highly-branched polymers are reacted with compounds A during and/or after the polymerization reaction. Compounds A are notable for the fact that they comprise a linear or branched C₄- to C₄₀-alkyl and/or alkenyl radical and a reactive group. A reactive group of compound A is able to react with the highly-branched polymer. Preferably, compounds A comprise precisely one linear or branched C₄- to C₄₀-alkyl and/or alkenyl radical and precisely one reactive group.

Highly-branched polymers which have been reacted with compounds A are referred to as substituted highly-branched polymers.

The substitution can take place completely or partially. This means in the case of complete substitution that the reactive groups of the highly-branched polymer have reacted completely with compounds A. In the case of partial substitution, not all of the reactive groups of the highly-branched polymer have reacted with compounds A.

Preferably, the highly-branched polymers are substituted by octyl(capryl), nonyl, decyl(caprinyl), undecyl, dodecyl(laurinyl), tetradecyl, hexadecyl(palmityl), heptadecyl, octadecyl(stearyl) radicals and/or the corresponding mono- or polyunsaturated equivalents, such as, for example, by dodecenyl, hexadienyl(sorbinyl), octadecenyl(oleyl), linolyl or linolenyl radicals.

In this connection, equivalent is to be understood as meaning a hydrocarbon radical which differs from the corresponding linear or branched alkyl radical only by virtue of the fact that it has at least one double bond.

The substituted highly-branched polycarbonates are preferably obtained by reacting the resulting high-functionality highly- or hyperbranched polycarbonate with a suitable functionalization reagent which can react with the OH and/or carbonate or carbamoyl chloride groups of the polycarbonate.

High-functionality highly-branched polycarbonates comprising hydroxyl groups can be modified, for example, by adding acid derivative groups, such as esters, anhydrides or amides or molecules comprising isocyanate groups. For example, polycarbonates comprising acid groups can be obtained through reaction with compounds comprising anhydride groups.

Here, the molar ratio of the reactive groups of the substitution compound to the reactive groups of the highly-branched polymer is from 1:10 to 1:1, preferably from 1:5 to 1:1.1, especially preferably from 1:2 to 1:1.2. A particularly preferred range is 1:1.7 to 1:1.4.

In one preferred embodiment of the present invention, the substitution takes place with a carboxylic acid derivative of the formula R—CO—Y and/or an isocyanate of the formula R—NCO, where the radicals have the meaning below.

R is linear or branched C₄- to C₄₀-alkyl.

Y is OR¹, OC(O)R² or NR³₂. Here, R¹ is hydrogen or linear or branched C₁- to C₆-alkyl,

R² is linear or branched C₄- to C₄₀-alkyl, where R and R² may be identical or different.

R³ is hydrogen or linear or branched C₁- to C₄-alkyl, where the two radicals R³ may be identical or different from one another.

Preferred compounds are linear C₄-C40-alkyl isocyanates, particular preference being given to octyl(capryl)isocyanate, nonyl isocyanate, decyl(caprinyl)isocyanate, undecyl isocyanate, dodecyl(laurinyl)isocyanate, tetradecyl isocyanate, hexadecyl(palmityl)isocyanate, heptadecyl isocyanate, octadecyl(stearyl)isocyanate.

Further preferred compounds are linear C₄-C₄₀-alkenyl isocyanates with one or more double bonds, particular preference being given to dodecenyl, hexadienyl(sorbinyl), octadecenyl(oleyl), linolyl or linolenyl isocyanate.

A very particularly preferred compound is stearyl isocyanate. The substitution can take place, for example, in a subsequent process step (step c)). However, the substitution can also take place as early as during the preparation of the highly-branched polymers.

Preferably, the substitution takes place in a subsequent process step.

If the substitution takes place in a subsequent process step, then preferably the highly-branched polycarbonate is initially introduced and one or more compounds A are added.

The substitution usually takes place at a temperature from 0 to 300° C., preferably 0 to 250° C., particularly preferably at 60 to 200° C. and very particularly preferably at 60 to 160° C. without a diluent or in solution. Here, in general it is possible to use all solvents which are inert towards the particular starting materials. Preference is given to using organic solvents, such as, for example, decane, dodecane, benzene, toluene, chlorobenzene, xylene, dimethylformamide, dimethylacetamide or solvent naphtha.

In one preferred embodiment, the substitution reaction is carried out without a diluent. In order to increase the rate of the reaction, low molecular weight compounds that are released during the reaction can be removed from the reaction equilibrium, for example by distillation, if necessary under reduced pressure.

To complete the reaction, it may be necessary to raise the temperature of the reaction container following the addition of compound A or, if two or more different compounds A are used, following the addition of compounds A. The increase is usually 10 to 50° C., it is preferably 20 to 40° C.

The substitution of the high-functionality polycarbonates in most cases takes place in a pressure range from 0.1 mbar to 20 bar, preferably at 1 bar to 5 bar, in reactors or reactor cascades which are operated in batch operation, semicontinuously or continuously.

The invention provides a cosmetic composition comprising at least one highly-branched polycarbonate.

Preferably, the highly-branched polycarbonate is substituted.

The cosmetic composition preferably comprises at least one cosmetically suitable carrier.

The use of a highly-branched polycarbonate in cosmetic and/or dermatological formulations is in accordance with the invention.

Preferably, the highly-branched polycarbonate is substituted.

Preferably, the use is in skin cosmetic formulations.

Preference is given to using a highly-branched polycarbonate as thickener. In this connection, in particular the use as oil thickener is preferred.

Skin cosmetic preparations

Skin cosmetic compositions according to the invention, in particular those for skincare, may be present and used in various forms. Thus, for example, they may be an emulsion of the oil-in-water (O/W) type or a multiple emulsion, for example of the water-in-oil-in-water (W/O/W) type. Emulsifier-free formulations such as hydrodispersions, hydrogels or a Pickering emulsion are also advantageous embodiments.

The consistency of the formulations can range from pasty formulations via flowable formulations to low viscosity, sprayable products. Accordingly, creams, lotions or sprays can be formulated. For use, the cosmetic compositions according to the invention are applied in an adequate amount to the skin in the manner customary for cosmetics and dermatological compositions.

The salt content in the surface of the skin is sufficient to lower the viscosity of the preparations according to the invention in such a way as to facilitate simple spreading and working-in of the preparations.

The skin cosmetic preparations according to the invention are present in particular as W/O or O/W skin creams, day and night creams, eye creams, face creams, antiwrinkle creams, mimic creams, moisturizing creams, bleaching creams, vitamin creams, skin lotions, care lotions and moisturizing lotions.

Further advantageous skin cosmetic preparations are face toners, face masks, deodorants and other cosmetic lotions and preparations for decorative cosmetics, for example concealing sticks, stage make-up, mascara, eyeshadows, lipsticks, kohl pencils, eyeliners, make-ups, foundations, blushers, powders and eyebrow pencils. Moreover, the compositions according to the invention can be used in nose strips for pore cleansing, in antiacne compositions, repellants, shaving compositions, hair removal compositions, intimate care compositions, foot care compositions, and in baby care. Besides the W/W emulsion polymer and suitable carriers, the skin cosmetic preparations according to the invention also comprise further active ingredients and/or auxiliaries customary in cosmetics, as described above and below.

These include preferably emulsifiers, preservatives, perfume oils, cosmetic active ingredients, such as phytantriol, vitamin A, E and C, retinol, bisabolol, panthenol, natural and synthetic photoprotective agents, bleaches, colorants, tinting agents, tanning agents, collagen, protein hydrolyzates, stablizers, pH regulators, dyes, salts, thickeners, gel formers, consistency regulators, silicones, humectants, conditioners, refatting agents and further customary additives.

Further polymers may also be added to the compositions if specific properties are to be set. To establish certain properties, such as, for example, improving the feel to the touch, the spreading behavior, the water resistance and/or the binding of active ingredients and auxiliaries such as pigments, the compositions can additionally also comprise conditioning substances based on silicone compounds. Suitable silicone compounds are, for example, polyalkylsiloxanes, polyarylsiloxanes, polyarylalkylsiloxanes, polyether siloxanes or silicone resins.

Further possible ingredients of the compositions according to the invention are described below under the respective keyword.

Oils, Fats and Waxes

The skin and hair cosmetic compositions preferably also comprise oils, fats or waxes. Constituents of the oil phase and/or fatty phase of the cosmetic compositions are advantageously selected from the group of lecithins and fatty acid triglycerides, namely the triglycerol esters of saturated and/or unsaturated, branched and/or unbranched alkanecarboxylic acids of chain length from 8 to 24, in particular 12 to 18, carbon atoms. The fatty acid triglycerides can, for example, be advantageously selected from the group of synthetic, semisynthetic and natural oils, such as, for example, olive oil, sunflower oil, soybean oil, peanut oil, rapeseed oil, almond oil, palm oil, coconut oil, castor oil, wheatgerm oil, grapeseed oil, thistle oil, evening primrose oil, macadamia nut oil and the like. Further polar oil components can be selected from the group of esters of saturated and/or unsaturated, branched and/or unbranched alkanecarboxylic acids of chain length from 3 to 30 carbon atoms and saturated and/or unsaturated, branched and/or unbranched alcohols of chain length from 3 to 30 carbon atoms, and also from the group of esters of aromatic carboxylic acids and saturated and/or unsaturated, branched and/or unbranched alcohols of chain length from 3 to 30 carbon atoms. Such ester oils can then advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, dicaprylyl carbonate (Cetiol CC) and cocoglycerides (Myritol 331), butylene glycol dicaprylate/dicaprate and dibutyl adipate, and also synthetic, semisynthetic and natural mixtures of such esters, such as, for example, jojoba oil.

In addition, one or more oil components can be advantageously selected from the group of branched and unbranched hydrocarbons and hydrocarbon waxes, the silicone oils, the dialkyl ethers, the group of saturated or unsaturated, branched or unbranched alcohols.

Any desired mixtures of such oil and wax components are also to be used advantageously within the context of the present invention. It may in some instances also be advantageous to use waxes, for example cetyl palmitate, as the sole lipid component of the oil phase.

According to the invention, the oil component is advantageously selected from the group consisting of 2-ethylhexyl isostearate, octyldodecanol, isotridecyl isononanoate, isoeicosane, 2-ethylhexyl cocoate, C12-15-alkyl benzoate, capryl-capric triglyceride, dicaprylyl ether.

Mixtures of C12-C15-alkyl benzoate and 2-ethylhexyl isostearate, mixtures of C12-C15-alkyl benzoate and isotridecyl isononanoate, and also mixtures of C12-C15-alkyl benzoate, 2-ethylhexyl isostearate and isotridecyl isononanoate are advantageous in accordance with the invention.

According to the invention, as oils with a polarity of from 5 to 50 mN/m, particular preference is given to using fatty acid triglycerides, in particular soybean oil and/or almond oil.

Of the hydrocarbons, paraffin oil, squalane, squalene and in particular polyisobutenes, which may also be hydrogenated, are to be used advantageously within the context of the present invention.

In addition, the oil phase can be advantageously selected from the group of Guerbet alcohols. Guerbet alcohols are produced by the reaction equation

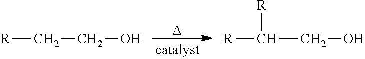

by oxidation of an alcohol to give an aldehyde, by aldol condensation of the aldehyde, elimination of water from the aldol and hydrogenation of the allylaldehyde. Guerbet alcohols are liquid even at low temperatures and cause virtually no skin irritations. They can be used advantageously as fatting, superfatting and also refatting constituents in cosmetic compositions.

The use of Guerbet alcohols in cosmetics is known per se. Such species are then characterized in most cases by the structure

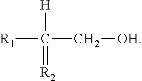

Here, R₁ and R₂ are generally unbranched alkyl radicals.

According to the invention, the Guerbet alcohol or alcohols are advantageously selected from the group where

R₁=propyl, butyl, pentyl, hexyl, heptyl or octyl and

R₂=hexyl, heptyl, octyl, nonyl, decyl, undecyl, dodecyl, tridecyl or tetradecyl.

Guerbet alcohols preferred according to the invention are 2-butyloctanol (commercially available, for example, as Isofol®12 (Condea)) and 2-hexyldecanol (commercially available, for example, as Isofol®16 (Condea)).

Mixtures of Guerbet alcohols according to the invention are also to be used advantageously according to the invention, such as, for example, mixtures of 2-butyl-octanol and 2-hexyldecanol (commercially available, for example, as Isofol®14 (Condea)).

Any desired mixtures of such oil and wax components are also to be used advantageously within the context of the present invention. Among the polyolefins, polydecenes are the preferred substances.

The oil component can advantageously also have a content of cyclic or linear silicone oils or consist entirely of such oils, although it is preferred to use an additional content of other oil phase components besides the silicone oil or the silicone oils.

Low molecular weight silicones or silicone oils are generally defined by the following general formula

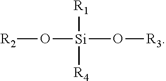

Higher molecular weight silicones or silicone oils are generally defined by the following general formula

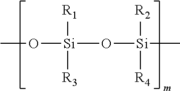

where the silicon atoms can be substituted by identical or different alkyl radicals and/or aryl radicals, which are shown here in general terms by the radicals R₁ to R₄. However, the number of different radicals is not necessarily limited to 4. m can here assume values from 2 to 200 000.

Cyclic silicones to be used advantageously according to the invention are generally defined by the following general formula

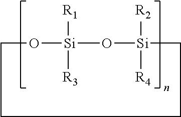

where the silicon atoms can be substituted by identical or different alkyl radicals and/or aryl radicals, which are represented here in general terms by the radicals R₁ to R₄.

However, the number of different radicals is not necessarily limited to 4. n can here assume values from 3/2 to 20. Fractional values for n take into consideration that odd numbers of siloxyl groups may be present in the cycle.

Phenyltrimethicone is advantageously selected as silicone oil. Other silicone oils, for example dimethicone, hexamethylcyclotrisiloxane, phenyldimethicone, cyclomethicone (e.g. decamethylcyclopentasiloxane), hexamethylcyclotrisiloxane, polydimethylsiloxane, poly(methylphenylsiloxane), cetyldimethicone, behenoxydimethicone are also to be used advantageously within the context of the present invention. Also advantageous are mixtures of cyclomethicone and isotridecyl isononanoate, and also those of cyclomethicone and 2-ethylhexyl isostearate.

However, it is also advantageous to select silicone oils of similar constitution to the compounds referred to above, the organic side chains of which are derivatized, for example polyethoxylated and/or polypropoxylated. These include, for example, polysiloxanepolyalkyl-polyether copolymers, such as, for example, cetyl-dimethicone copolyol.

Cyclomethicone(octamethylcyclotetrasiloxane) is advantageously used as silicone oil to be used according to the invention.

Fatty and/or wax components to be used advantageously can be selected from the group of vegetable waxes, animal waxes, mineral waxes and petrochemical waxes. For example, candelilla wax, carnauba wax, Japan wax, esparto grass wax, cork wax, guaruma wax, rice germ oil wax, sugar cane wax, berry wax, ouricury wax, montan wax, jojoba wax, shea butter, beeswax, shellac wax, spermaceti, lanolin (wool wax), uropygial grease, ceresin, ozokerite (earth wax), paraffin waxes and micro waxes. Further advantageous fatty and/or wax components are chemically modified waxes and synthetic waxes, such as, for example, Syncrowax®HRC (glyceryl tribehenate), and Syncrowax®AW 1 C (C₁₈₋₃₆-fatty acid), and also montan ester waxes, sasol waxes, hydrogenated jojoba waxes, synthetic or modified beeswaxes (e.g. dimethicone copolyol beeswax and/or C₃₀₋₅₀-alkyl beeswax), cetyl ricinoleates, such as, for example Tegosoft®CR, polyalkylene waxes, polyethylene glycol waxes, but also chemically modified fats, such as, for example, hydrogenated plant oils (for example hydrogenated castor oil and/or hydrogenated coconut fatty glycerides), triglycerides, such as, for example, hydrogenated soy glyceride, trihydroxystearin, fatty acids, fatty acid esters and glycol esters, such as, for example, C₂₀₋₄₀-alkyl stearate, C₂₀₋₄₀-alkyl hydroxystearoyl stearate and/or glycol montanate. Also certain organosilicon compounds which have similar physical properties to the specified fat and/or wax components, such as, for example, stearoxytrimethylsilane, are further advantageous. According to the invention, the fat and/or wax components can be used either individually or as a mixture in the compositions.

Any desired mixtures of such oil and wax components are also to be used advantageously within the context of the present invention.

The oil phase is advantageously selected from the group consisting of 2-ethylhexyl isostearate, octyldodecanol, isotridecyl isononanoate, butylene glycol dicaprylate/dicaprate, 2-ethylhexyl cocoate, C₁₂₋₁₅-alkyl benzoate, caprylic-capric triglyceride, dicaprylyl ether.

Mixtures of octyldodecanol, caprylic-capric triglyceride, dicaprylyl ether, dicaprylyl carbonate, cocoglycerides or mixtures of C₁₂₋₁₅-alkyl benzoate and 2-ethylhexyl isostearate, mixtures of C₁₂₋₁₅-alkyl benzoate and butylene glycol dicaprylate/dicaprate, and also mixtures of C₁₂₋₁₅-alkyl benzoate, 2-ethylhexyl isostearate and isotridecyl isononanoate are particularly advantageous.

Of the hydrocarbons, paraffin oil, cycloparaffin, squalane, squalene, hydrogenated polyisobutene and polydecene are to be used advantageously within the context of the present invention.

The oil component can also be advantageously selected from the group of phospholipids. The phospholipids are phosphoric acid esters of acylated glycerols. Of greatest importance among the phosphatidylcholines are, for example, the lecithins, which are characterized by the general structure

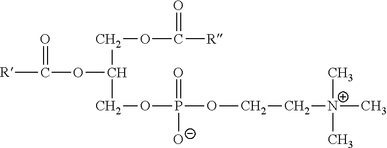

where R′ and R″ are typically unbranched aliphatic radicals having 15 or 17 carbon atoms and up to 4 cis double bonds.

According to the invention, as paraffin oil advantageous according to the invention it is possible to use Merkur Weissoel Pharma 40 from Merkur Vaseline, Shell Ondina® 917, Shell Ondina® 927, Shell Oil 4222, Shell Ondina®933 from Shell & DEA Oil, Pionier® 6301 S, Pionier® 2071 (Hansen & Rosenthal).

Suitable cosmetically compatible oil and fat components are described in Karl-Heinz Schrader, Grundlagen and Rezepturen der Kosmetika [Fundamentals and formulations of cosmetics], 2nd edition, Verlag Hüthig, Heidelberg, pp. 319-355, to which reference is hereby made in its entirety.

Further embodiments of the present invention are given in the claims, the description and the examples. It goes without saying that the features of the subject matter according to the invention that have been specified above and are still to be explained below can be used not only in the combination stated in each case, but also in other combinations, without departing from the scope of the invention.

The present invention will be illustrated by the examples below.

### EXAMPLES

Measurement Methods

The IR measurements were carried out using a Nicolet 210 instrument.

The hydroxyl number was determined in accordance with DIN 53240, part 2.

The molecular weight was determined with the help of gel permeation chromatography using a refractometer as detector. The mobile phase used was dimethylacetamide, and the standard used for determining the molecular weight was polymethyl methacrylate (PMMA).

Feed Materials

DBTL: dibutyltin dilaurate, manufacturer: Sigma-Aldrich

Aluminum chlorohydrate: activated Aloxicoll® powder, manufacturer: Giulini, Ludwigshafen, Germany

Hydrogenated polyisobutene: Luvitol® Lite, manufacturer: BASF Aktiengesellschaft, Ludwigshafen, Germany

Paraffin oil: Nujol, Fluka AG

### Example 1

### Preparation of a Highly-Branched Polycarbonate

88.6 g of diethyl carbonate (0.75 mol) and 150 g (0.75 mol) of a triol based on trimethylpropane which has been etherified in a random manner with 1,2-propylene oxide units were initially introduced into a 500 ml glass reactor equipped with stirrer, reflux condenser, gas inlet, attached cold trap and internal thermometer. Following the addition of 0.02 g of potassium carbonate, the mixture was heated to 120° C., and stirred for 3 h at this temperature. The ethanol which formed was distilled off (46 g). When the distillation was complete, 0.01 g of phosphoric acid were added to neutralize the catalyst and the mixture was stirred for one hour at 100° C. The temperature of the reaction mixture was then increased to 120° C. and the remaining ethanol was stripped off under a stream of nitrogen.

The end product was filtered over a 125 μm filter and obtained as a clear, colorless low viscosity resin which has the following properties: hydroxyl number=451 mg KOH/g;

Mn=1300 g/mol, Mw=2000 g/mol.

### Examples 2-9

### Modification of the Highly-Branched Polycarbonate with Stearyl Isocyanate

Highly-branched polycarbonate from Example 1 was initially introduced into a 250 ml glass reactor equipped with stirrer, reflux condenser, gas inlet, internal thermometer and dropping funnel which comprised the required amount of stearyl isocyanate. The amounts of highly-branched polycarbonate and stearyl isocyanate used are given in the table below.

The reactor was heated to 100° C. and the isocyanate was added dropwise over the course of 15 minutes. The reaction mixture was then stirred for a further three hours at 130° C. and the reaction progress was monitored via the disappearance of the isocyanate groups with the help of IR spectroscopy (vibration of the isocyanate band at 2270 cm⁻¹).

| | Example | | | | | | | |
|---|---|---|---|---|---|---|---|---|
| | 2 | 3 | 4 | 5 | 6 | 7 | 8 | 9 |
| Highly- | 100 | 100 | 100 | 76 | 50 | 50 | 50 | 50 |
| branched | | | | | | | | |
| polycarbonate | | | | | | | | |
| (Example 1) | | | | | | | | |
| [g] | | | | | | | | |
| Mol % NCO | 10 | 20 | 30 | 50 | 60 | 70 | 90 | 100 |
| Amount of | 23.8 | 47.6 | 71.4 | 90.1 | 71.4 | 83.3 | 107.1 | 119 |
| stearyl | | | | | | | | |
| isocyanate [g] | | | | | | | | |

### Example 10

### Gel Formation by Adding the Stearyl-Modified Highly-Branched Polycarbonates to Paraffin Oil

Various amounts (0.5 to 40% by weight) of the polymers of Examples 2 to 9 were dissolved in paraffin oil. The concentration at which a visible gel formation occurred is given in the table below:

| | Polymer from example . . . | | | | | | | |
|---|---|---|---|---|---|---|---|---|
| | 2 | 3 | 4 | 5 | 6 | 7 | 8 | 9 |
| NCO/OH ratio [%] | 10 | 20 | 30 | 50 | 60 | 70 | 90 | 100 |
| Gel formation | 30 | 10 | 5 | 1.5 | 1 | 1 | 2.5 | 5 |
| concentration [%] | | | | | | | | |

### Example 11

### Preparation of a Deodorant Stick Based on Hydrogenated Polyisobutene Oil

4 g of the polycarbonate from Example 7 and 20 g of aluminum chlorohydrate are mixed with 76 g of hydrogenated polyisobutene oil at 80° C. with stirring. Following complete dissolution of the polycarbonate, the mixture is poured into a deodorant stick mold and cooled to ambient temperature. The finished deodorant stick is a waxy product that is solid at ambient temperature.

### Example 12

### Preparation of a Deodorant Stick Based on Paraffin Oil

4 g of the polycarbonate from Example 7 and 20 g of aluminum chlorohydrate are mixed with 76 g of paraffin oil at 80° C. with stirring. Following complete dissolution of the polycarbonate, the mixture is poured into a deodorant stick mold and cooled to ambient temperature. The finished deodorant stick is a waxy product that is solid at ambient temperature. The viscosity of the product is 60 Pa s (20° C.). In the course of the measurement, the value drops to 30 Pa s.

1-10. (canceled)

11. A cosmetic or dermatological composition comprising at least one highly-branched polycarbonate which is substituted completely or partly with linear or branched C₄-C₄₀-alkyl or alkenyl radicals, wherein the substitution takes place with a compound of the formula R—CO—Y and/or R—NCO
where
R is linear or branched C₄-C₄₀-alkyl,
Y is OR¹, OC(O)R², NR³₂, or halogen,
R¹ is hydrogen, linear or branched C₁-C₆-alkyl,
R² is linear or branched C₄-C₄₀-alkyl, where R and R² may be identical or different,
R³ are hydrogen, linear or branched C₁-C₄-alkyl, where the two radicals R³ may be identical or different from one another.

12. A cosmetic formulation which comprises the composition as claimed in claim 11.

13. A dermatological formulation which comprises the composition as claimed in claim 11.

14. The cosmetic composition according to claim 11, comprising at least one cosmetically suitable carrier.

15. The cosmetic formulation according to claim 12, wherein the cosmetic formulation is a skin cosmetic formulation.

16. A thickener which comprises the at least one highly-branched polycarbonate according to claim 11.

17. An oil thickener which comprises the at least one highly-branched polycarbonate according to claim 11.

18. The composition as claimed in claim 11, wherein the compound of the formula R—NCO is selected from the group consisting of octyl isocyanate, nonyl isocyanate, decyl isocyanate, undecyl isocyanate, dodecyl isocyanate, tetradecyl isocyanate, hexadecyl isocyanate, heptadecyl isocyanate, and octadecyl isocyanate.

19. The composition as claimed in claim 11, wherein the compound of the formula R—NCO is octadecyl isocyanate.
